(19) United States
(12) Patent Application Publication
(10) Pub. No.: US 2010/0278907 A1
(43) Pub. Date: Nov. 4, 2010

(54) Immunogenic Compositions Containing Ceramide and Methods of Use Thereof

(75) Inventor: Erhard Bieberich, Augusta, GA (US)

Correspondence Address: Pabst Patent Group LLP, 1545 PEACHTREE STREET NE, SUITE 320, ATLANTA, GA, 30309, US

(73) Assignee: Medical College of Georgia Research Institute, Inc

(21) Appl. No.: 12/778,523

(22) Filed: May 12, 2010

Related U.S. Application Data
(63) Continuation of application No. PCT/US2009/041380, filed on Apr. 22, 2009.
(60) Provisional application No. 61/125,126, filed on Apr. 22, 2008.

(51) Int. Cl. A61K 38/19; A61K 39/00; A61K 39/385; A61K 9/127; A61K 38/20; A61K 38/21; A61P 11/00; A61P 25/00; A61P 31/04; A61P 9/00; A61P 9/10

(52) U.S. Cl. 424/450; 424/184.1; 424/193.1; 424/195.11; 424/197.11; 424/85.2; 424/85.4; 424/85.1

(57) ABSTRACT

Immunogenic compositions containing ceramide or ceramide analogs for treating or reducing the risk of developing one or more symptoms of a disease or disorder associated with ceramide-induced cell death are provided. The immunogenic compositions contain immunogenic ceramide, and, optionally, pharmaceutically acceptable excipients and one or more additional adjuvants. Methods of using the disclosed immunogenic ceramide compositions for reducing ceramide-induced cell death are provided. Methods of using the disclosed immunogenic ceramide compositions therapeutically or prophylactically for treating or reducing the risk of developing one or more symptoms of a disease or disorder associated with ceramide-induced cell death are also provided.

### CROSS REFERENCE TO RELATED APPLICATIONS

This application claims priority to and benefit of U.S. Provisional Patent Application No. 61/125,126, filed on Apr. 22, 2008, by Erhard Bieberich, and where permissible is incorporated by reference in its entirety.

### STATEMENT REGARDING FEDERALLY SPONSORED RESEARCH OR DEVELOPMENT

This invention was made with government support awarded by the National Institutes of Health under Grant Number NIH R01NS046835 to Erhard Bieberich. The United States government has certain rights in this invention.

### FIELD OF THE INVENTION

The present disclosure generally relates to the field of immunogenic ceramide compositions and methods of use thereof.

### BACKGROUND OF THE INVENTION

Ceramides are a family of lipid molecules that contain sphingosine and a fatty acid. Ceramide synthesis can occur through a de novo pathway, through hydrolysis of sphingomyelin or through a salvage pathway in which complex sphingolipids are broken down into sphingosine with is then used to form ceramide through acylation.

De novo synthesis of ceramide occurs in the endoplasmic reticulum. The first step in de novo synthesis of ceramide occurs through the condensation of palmitate and serine to form 3-keto-dihydrosphingosine through the action of serine palmitoyl transferase. 3-keto-dihydrosphingosine is then reduced to dihydrosphingosine which is then followed by acylation through the action of (dihydro)ceramide synthase to produce dihydroceramide. The final reaction to produce ceramide is catalyzed by dihydroceramide destaurase. Ceramide is then transported from the endoplasmic reticulum to the Golgi either be vesicular trafficking or the ceramide transfer protein CERT. Once in the Golgi apparatus, ceramide can be further metabolized to other sphingolipids, such as sphingomyelin and the complex glycosphingolipids. The major route of sphingomyelin synthesis occurs through the donation of the phophocholine group of phophatidylchline to ceramide.

Ceramide can also be formed from the hydrolysis of sphingomyelin catalyzed by the enzyme sphingomyleinase (SMase). SMases have been characterized as acid SMase, secretory SMase, neutral Mg²⁺ dependent SMase, Mg²⁺ independent neutral SMase and alkaline SMase. Although de novo synthesis and production from sphingomyuelin hydrolysis are the two major pathways for ceramide synthesis, ceramide can also be produced by the breakdown of complex sphingolipids into sphingosine with is then used to form ceramide through acylation.

Ceramide has been shown in recent years to be involved in cellular apoptosis and stress responses. In particular, ceramide generated by the activity of the acid SMase has been shown by many studies to play a pivotal role in the immediate stress response and in apoptotic stimuli in almost any mammalian cell, while ceramide synthases seem to act slower (Gulbins and Kolesnick, Oncogene, 22:7070-7077 (2003). The acid SMase is activated by many stimuli including CD95, CD40, DR5/TRAIL, CD20, FcγRII, CD5, LFA-1, CD28, TNFα, Interleukin-1 receptor, PAF-receptor, infection with Pseudomonas aeruginosa, Staphylococcus aureus, Neisseria gonorrhoeae, γ-irradiation, UV-light, doxorubicin, cisplatin, gemcitabine, paclitaxel, disruption of integrin-signaling, amyloid peptides and some conditions of developmental death.

The proapoptotic effects of ceramide are mediated by a variety of mechanisms, including activation of the kinase suppressor of Ras, protein phosphatases 1 and 2A, cathepsin D, or through direct alteration of plasma or mitochondrial membrane signaling properties. Most of the above-mentioned stimuli trigger a translocation of the acid SMase onto the extracellular leaflet of the cell membrane upon stimulation resulting in the release of ceramide in the outer leaflet of the cell membrane. Ceramide molecules reorganize the cell membrane leading to the formation of large, distinct ceramide-enriched membrane domains that serve to cluster and aggregate activated receptor molecules. The formation of these ceramide-enriched membrane domains seems to be mediated by the tendency of ceramide molecules to associate with each other and to form ceramide-enriched microdomains that spontaneously fuse to large ceramide-enriched membrane platforms. Furthermore, ceramide seems to replace cholesterol in those membrane domains resulting in a fundamental change of membrane properties in these ceramide-enriched membrane domains.

Several studies have now shown a direct role for ceramide in the development of a variety of diseases and disorders. For example, Petrache, et al. demonstrated a role for ceramide in emphysema using a rat and mouse models (Petrache, et al., Nature Medicine, 11(5):491-8 (2005). This study demonstrated elevation of lung ceramide levels in individuals with smoking-induced emphysema. Emphysema was produced in rats and mice by installation of ceramide, and ceramide-specific antibodies decreased lung ceramides and attenuated lung apoptosis in these models. This study also suggested that a feedforward mechanism mediated by activation of the secretory ASMase is involved in the development of emphysema. Ceramide has also been implicated in the development of several neurological diseases and radiation-induced injury (Kolesnick and Fuks, Oncogene, 22:5897-5906 (2003); Luberto, et al., Neurochem. Res., 27:609-17 (2002)).

Despite the growing recognition of the role of ceramide in human diseases, treatments to reduce ceramide levels are largely lacking. Existing antibodies that bind to ceramide are either of mouse or rabbit origin and are not suitable for use in humans. Further, direct administration of antibodies as therapeutic molecules is a limited approach because it is only transiently effective and its effect is titratable.

Therefore, it is an object of the invention to provide compositions and methods for reducing levels of extracellular ceramide or inhibitiong one or more biological activities of extracellular ceramide in subjects.

It is another object of the invention to provide compositions and methods for reducing ceramide-induced cell death.

It is yet another object of the invention to provide compositions and methods for treating or preventing diseases or disorders associated with ceramide-induced cell death.

### SUMMARY OF THE INVENTION

Immunogenic compositions containing ceramide or ceramide analogs are provided. The immunogenic compositions contain immunogenic ceramide and, optionally, pharmaceutically acceptable excipients and one or more additional adjuvants.

Immunogenic ceramides or ceramide analogs include naturally occurring ceramides or ceramide analogs that have been modified to have increased immunogenicity relative to the ceramide or ceramide analog in the absence of the modification. Suitable modifications include changes in the chemical structure of the ceramide or ceramide analog, or association of the ceramide or ceramide analog with an immunogenic carrier molecule.

The immunogenic compositions can contain any combination of one or more species of immunogenic naturally occurring ceramide or ceramide analog. Suitable naturally occurring ceramides include, but are not limited to, C2 ceramide, C8 ceramide, C16 ceramide, C18 ceramide, C20 ceramide and C24 ceramide. Many suitable ceramide analogs are known in the art. Particularly preferred ceramide analogs include analogs that have increased immunogenicity as compared to naturally occurring ceramides.

Many suitable immunogenic carrier molecules are known in the art. In a preferred embodiment, the carrier molecule is keyhole limpet hemocyanin (KLH). The ceramide or ceramide analog can be covalently or non-covalently attached to the carrier molecule.

The immunogenic ceramide compositions can optionally contain one or more additional adjuvants. Many suitable adjuvants are known in the art.

The immunogenic ceramide formulations can optionally also contain pharmaceutically acceptable excipients. The disclosed immunogenic ceramide formulations can be formulated for parenteral (intramuscular, intraperitoneal, intravenous or subcutaneous injection), enteral, transdermal, or transmucosal routes of administration. The formulations can be formulated in unit dosage forms for ease of administration and uniformity of dosage. Suitable unit dosage forms include unit-dose or multi-dose containers, such as sealed ampules and vials.

Antibodies that specifically bind to ceramide that are generated using the disclosed immunogenic ceramide compositions are also disclosed. The antibodies can be monoclonal or polyclonal antibodies and can be xenogeneic, allogeneic, syngeneic, or modified forms thereof, such as humanized or chimeric antibodies.

Methods for using the immunogenic ceramide compositions to induce an immune response in a subject are provided. One embodiment provides a method for administering the immunogenic ceramide compositions to a subject to induce a humoral immune response including the production of anti-ceramide antibodies effective to bind to and reduce levels of extracellular ceramide and/or to inhibit one or more biological activities of extracellular ceramide.

In another embodiment, the disclosed immunogenic ceramide compositions or anti-ceramide antibodies are administered to an individual in an effective amount to inhibit or reduce or reduce the risk of ceramide-induced cell death in a subject.

In another embodiment, the disclosed immunogenic ceramide compositions or anti-ceramide antibodies are administered to an individual in an effective amount to treat or reduce the risk of developing one or more symptoms of a disease or disorder associated with ceramide-induced cell death. The disclosed immunogenic ceramide compositions or anti-ceramide antibodies can be administered therapeutically or prophylactically.

Diseases and disorders associated with ceramide-induced cell death are known in the art and include, but are not limited to, pulmonary diseases and disorders, neurological diseases and disorders, cardiovascular diseases and disorders, ischemic diseases and disorders and infectious diseases.

Any acceptable method known to one of ordinary skill in the art can be used to administer the disclosed immunogenic ceramide formulations or anti-ceramide antibodies to a subject. In general, methods of administering immunogenic formulations and antibodies are well known in the art. The administration can be localized (i.e., to a particular region, physiological system, tissue, organ, or cell type) or systemic.

The disclosed immunogenic ceramide compositions or anti-ceramide antibodies can be administered alone or in combination with one or more additional therapeutic or prophylactic agents. Suitable additional agents include, but are not limited to, anti-inflammatory and anti-apoptotic agents.

### BRIEF DESCRIPTION OF THE DRAWINGS

FIG. 1 is a line graph showing that anti-ceramide rabbit IgG specifically recognizes ceramide in lipid overlay assays. The graph shows that anti-ceramide rabbit IgG binds to ceramide (-•-) significantly more strongly than to sphingomyelin (SM) (-◯-) or phosphatidylcholine (PC) (-Δ-). Data are expressed as optical density (O.D. 492) as a function of dilution factor.

FIG. 2 is a bar graph showing a reduction in ceramide levels in F11 cells using the serine palmitoyl transferase inhibitor myriocin indicating the specificicity of the anti-ceramide antibody for ceramide. Data are expressed as fluorescence intensity (% of control).

### DETAILED DESCRIPTION OF THE INVENTION

### I. Definitions

As used herein the term “isolated” is meant to describe a compound of interest (e.g., a lipid or a polypeptide) that is in an environment different from that in which the compound naturally occurs, e.g., separated from its natural milieu such as by concentrating a peptide to a concentration at which it is not found in nature. “Isolated” is meant to include compounds that are within samples that are substantially enriched for the compound of interest and/or in which the compound of interest is partially or substantially purified.

As used herein, the terms “epitope” or “antigenic determinant” refer to a site on an antigen to which B and/or T cells respond.

As used herein, the terms “immunologic”, “immunological” or “immune” response is the development of a humoral (antibody mediated) and/or a cellular (mediated by antigen-specific T cells or their secretion products) response directed against an antigen. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response can also involve activation of monocytes, macrophages, NK cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

As used herein, a “costimulatory polypeptide” or a “costimulatory molecule” is a polypeptide that, upon interaction with a cell-surface molecule on T cells, enhances T cell responses, enhances proliferation of T cells, enhances production and/or secretion of cytokines by T cells, stimulates differentiation and effector functions of T cells or promotes survival of T cells relative to T cells not contacted with a costimulatory peptide.

As used herein, “treating” refers to both therapeutic treatment and prophylactic or preventative measures, wherein the object is to prevent or lessen one or more symptoms of, or the risk of developing one or more symptoms of, a disease associated with ceramide-induced cell death as described herein. Thus, in one embodiment, treating can include directly affecting or curing, suppressing, inhibiting, preventing, reducing the severity of, delaying the onset of, reducing symptoms associated with ceramide-induced cell death, or a combination thereof. Thus, in one embodiment, “treating” refers to delaying progression, expediting remission, inducing remission, augmenting remission, speeding recovery, increasing efficacy of or decreasing resistance to alternative therapeutics, or a combination thereof. In one embodiment, “preventing” refers to delaying the onset of symptoms, preventing relapse to a disease, decreasing the number or frequency of relapse episodes, increasing latency between symptomatic episodes, or a combination thereof. In one embodiment, “suppressing” or “inhibiting”, refers to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

The terms “individual”, “host”, “subject”, and “patient” are used interchangeably herein, and refer to a mammal, including, but not limited to, humans, rodents such as mice and rats, and other laboratory animals.

### II. Immunogenic Ceramide Compositions

Immunogenic compositions containing immunogenic ceramide useful to induce a humoral immune response in a subject are provided. The compositions are useful for reducing levels of extracellular ceramide and for reducing ceramide-induced cell death in a subject. The compositions can be used to treat one or more symptoms of a disease associated with ceramide-induced cell death or to reduce the risk of developing one or more symptoms of a disease associated with ceramide-induced cell death. The immunogenic compositions include immunogenic ceramide, and optionally, pharmaceutically acceptable excipients and one or more additional adjuvants.

A. Immunogenic Ceramide and Ceramide Analogs

The disclosed immunogenic compositions contain one or more species of immunogenic ceramides or ceramide analogs. The terms “immunogenic ceramides” or “immunogenic ceramides or ceramide analogs” refers to naturally occurring ceramides or ceramide analogs that have been modified to have increased immunogenicity relative to the ceramide or ceramide analog in the absence of the modification. Suitable modifications include changes in the chemical structure of the ceramide or ceramide analog, or association of the ceramide or ceramide analog with a second molecule that increases the immunogenicity of the ceramide or ceramide analog. For example, ceramides or ceramide analogs can be covalently or non-covalently attached to immunogenic carrier molecules, such as those described below.

Ceramides are a family of lipid molecules that contain sphingosine and a fatty acid. Naturally occurring ceramides differ in the length of their fatty acyl chains. Exemplary species of naturally occurring ceramides that can be included in the formulations include, but are not limited to, C2 ceramide, C8 ceramide, C16 ceramide, C18 ceramide, C20 ceramide and C24 ceramide.

Many ceramide analogs and ceramide mimetics are known in the art. Preferred ceramide analogs include analogs that have increased immunogenicity as compared to naturally occurring ceramides. Such analogs may be useful to increase the immune response of the host to the immunogenic formulation. Suitable ceramide analogs include, but are not limited to, C16-serinol and (2S, 3R)-(4E, 6E)-2-octanoylamidooctadecadiene-1,3-dial (4,6-dieneceramide) (Bieberich, et al., J. Biol. Chem., 275:177-181 (2000); Struckhoff, et al., J. Pharmacol. Exp. Ther., 309:523-532 (2004)), 5R-OH-3E-C8-ceramide, adamantyl-ceramide and benzene-C₄-ceramide (Crawford et al., Cell Mol. Biol., 49:1017-1023 (2003)). Other suitable ceramide analogs include those of the β-hydroxyalkylamine type, including those with saturated or mono- or polyunsaturated (cis or trans) alkyl groups. Exemplary ceramide analogs of this type include, but are not limited to N-(2-hydroxy-1-(hydroxymethyl)ethyl)-palmitoylamide (“S16”); N-(2-hydroxy-1-(hydroxymethyl)ethyl-oleoylamide (“S18”); N,N-bis(2-hydroxyethyl)palmitoylamide (“B16”); N,N-bis(2-hydroxyethyl)oleoylamide (“B18”); N-tris(hydroxymethyl)methyl-palmitoylamide (“T16”); N-tris(hydroxymethyl)methyl-oleoylamide (“T18”); N-acetyl sphingosine (“C2”); D-threo-1-phenyl-2-decanoylamino-3-morpholino-1 propanol (“D-threa-PDMP”); D-threo-1-phenyl-2-hexadecanoylamino-3-morpholino-1-propanol (“D-Threo-PPMP”); D-erythro-2-tetradecanoyl-1-phenyl-1-propanol (“D-MAPP”); D-erythro-2-(N-myristoylamino)-1-phenyl-1-propanol (“MAPP”); (1S, 2R)-D-erythro-2-(N-myristoylamino)-1-phenyl-1-propanol; and N-hexanoylsphingosine (C6-ceramide). Other suitable ceramide analogs include amino ceramide-like compounds that are described in U.S. Pat. No. 7,335,681 and U.S. Published Application No. 2008/0146533. Additional suitable ceramide analogs are provided in U.S. Pat. No. 5,631,394 and in Szulc, et al., Bioorg. Med. Chem., 14:7083-7104 (2006); Bielawska, et al., Bioorg. Med. Chem., 16:1032-1045 (2008) and; Senkal et al., J. Pharmacol. Exp. Ther., 317:1188-1199 (2006). Other suitable ceramide analogs include ceramides derivatized with polymers, such as poly(ethylene glycol) (PEG). Exemplary pegylated ceramides are described in Stover et al., Clin. Cancer Res., 11:3465-3474 (2005).

In one embodiment, the disclosed immunogenic ceramide compositions contain a single species of an immunogenic naturally occurring ceramide or ceramide analog. In a preferred embodiment, the disclosed immunogenic ceramide compositions contain a mixture of two or more species of immunogenic naturally occurring ceramides or ceramide analogs. For example, the immunogenic ceramide compositions can contain two or more species of naturally occurring ceramides, two or more species of ceramide analogs, or a mixture of at least one naturally occurring ceramide and at least one ceramide analog.

B. Immunogenic Carrier Molecules

In one embodiment, immunogenic ceramides or ceramide analogs include ceramides or ceramide analogs associated with one or more immunogenic carrier molecules. Small haptenic molecules can first be attached to an immunogenic carrier, such as a protein, to elicit a competent immune response (Williams and Chase, Eds., Methods in Immunology and Immunochemistry, vol. 1, pp 120-187 (1967); G. T. Hermanson, Bioconjugate Techniques, (Academic Press, New York, pp 419-455 (1996)). Naturally occurring ceramides or ceramide analogs used in the formulations can be covalently or non-covalently attached to the immunogenic carrier molecules.

Suitable immunogenic carrier molecules for use in the disclosed formulations include, but are not limited to, keyhole limpet hemocyanin (KLH), serum albumin, ovalbumin, thyroglobulin, toxoids derived from diphtheria and tetanus, bacteria outer membrane proteins, crystalline bacterial cell surface layers, various endo or exotoxins, gamma globulin, exotoxin A, L T toxin, Cholera B toxin, Klebsiella pneumoniae OmpA, Bacterial flagella, Clostridium difficile recombinant toxin A, peptide dendrimers (multiple antigenic peptides), and pan DR epitope (PADRE).

In a preferred embodiment, the immunogenic carrier molecule is KLH. KLH is an extremely large, heterogeneous glycosylated protein consisting of subunits with a molecular weight of 350,000 and 390,000 in aggregates with molecular weights of 4,500,000-13,000,000. Each domain of a KLH subunit contains two copper atoms that together bind a single oxygen molecule (O₂). When oxygen is bound to hemocyanin, the molecule takes on a distinctive transparent, opalescent blue color. KLH is potently immunogenic due to its structural features and large size, yet safe in humans. In addition, KLH generally forms particulate immunoconjugates that can further enhance immunogenicity.

Methods for conjugating molecules to KLH and to other carrier proteins are well known in the art. For example, a simple one-step coupling can be performed using the crosslinker 1-Ethyl-3[3-dimethylaminopropyl] carbodiimide hydrochloride (EDC) to covalently attach carboxyls to primary amines. Additional crosslinkers that mediate covalent attachment through reaction with other functional groups are known in the art.

C. Additional Adjuvants

Optionally, the disclosed immunogenic compositions can include one or more additional adjuvants. The adjuvant can be, but is not limited to, one or more of the following: oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; bacille Calmette-Guerin (BCG); mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, and dinitrophenol).

Additional adjuvants can also include immunomodulators such as cytokines, interleukins (e.g., IL-1, IL-2, IL-4, IL-5, IL-6, IL-7, IL-12, etc.), interferons (e.g., interferon-γ), macrophage colony stimulating factor, and tumor necrosis factor. Additional adjuvants can include costimulatory molecules, including, but not limited to polypeptides of the B7 family of costimulatory molecules, such as 137.1, B7.2, B7-DC (PD-L2), B7-H3 or B7-145. Such proteinaceous adjuvants can be provided as the full-length polypeptide or an active fragment thereof, or in the form of DNA, such as plasmid DNA.

D. Pharmaceutical Excipients

The immunogenic compositions disclosed herein can be combined with one or more pharmaceutically acceptable excipients. As would be appreciated by one of skill in this art, the excipients can be chosen based on the route of administration, including parenteral (intramuscular, intraperitoneal, intravenous (IV) or subcutaneous injection), enteral, transdermal (either passively or using iontophoresis or electroporation), or transmucosal (nasal, vaginal, rectal, or sublingual) routes of administration and can be formulated in dosage forms appropriate for each route of administration.

As used herein, the term “pharmaceutically acceptable excipient” means a non-toxic, inert solid, semi-solid or liquid filler, diluent, encapsulating material or formulation auxiliary of any type. Remington's Pharmaceutical Sciences Ed. by Gennaro, Mack Publishing, Easton, Pa., 1995 discloses various carriers used in formulating pharmaceutical compositions and known techniques for the preparation thereof.

Suitable excipients include surfactants, emulsifiers, emulsion stabilizers, anti-oxidants, emollients, humectants, chelating agents, suspending agents, thickening agents, occlusive agents, preservatives, stabilizing agents, pH modifying agents, solubilizing agents, solvents, colorants, fragrances, penetration enhancers, and other excipients.

### 1. Formulations for Parenteral Administration

In a preferred embodiment, compositions disclosed herein are administered in an aqueous solution, by parenteral injection. The formulations can be lyophilized and redissolved/resuspended immediately before use. The formulation can be sterilized by, for example, filtration through a bacteria retaining filter, by incorporating sterilizing agents into the compositions, by irradiating the compositions, or by heating the compositions. The formulation can also be in the form of a suspension or emulsion. In general, pharmaceutical compositions are provided including effective amounts of immunogenic ceramides or ceramide analogs, and optionally include pharmaceutically acceptable diluents, preservatives, antioxidants, chelating agents, pH modifying agents, solubilizers, emulsifiers, or carriers.

### i. Diluents

Diluents can be included in the formulations to dissolve, disperse or otherwise incorporate the immunogenic composition. Examples of diluents include, but are not limited to, water, buffered aqueous solutions, organic hydrophilic diluents, such as monovalent alcohols, and low molecular weight glycols and polyols (e.g. propylene glycol, polypropylene glycol, glycerol, butylene glycol).

### ii. Preservatives

Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, thimerosal, and combinations thereof.

### iii. Antioxidants

Suitable antioxidants include, but are not limited to, butylated hydroxytoluene, alpha tocopherol, ascorbic acid, fumaric acid, malic acid, butylated hydroxyanisole, propyl gallate, sodium ascorbate, sodium metabisulfite, ascorbyl palmitate, ascorbyl acetate, ascorbyl phosphate, Vitamin A, folic acid, flavons or flavonoids, histidine, glycine, tyrosine, tryptophan, carotenoids, carotenes, alpha-Carotene, beta-Carotene, uric acid, pharmaceutically acceptable salts thereof, derivatives thereof, and combinations thereof.

### iv. Chelating Agents

Suitable chelating agents include, but are not limited to, EDTA, disodium edetate, trans-1,2-diaminocyclohexane-N,N,N′,N′-tetraaceticacid monohydrate, N,N-bis(2-hydroxyethyl)glycine, 1,3-diamino-2-hydroxypropane-N,N,N′,N′-tetraacetic acid, 1,3-diaminopropane-N,N,N′,N′-tetraacetic acid, ethylenediamine-N,N′-diacetic acid, ethylenediamine-N,N′-dipropionic acid, ethylenediamine-N,N′-bis(methylenephosphonic acid), N-(2-hydroxyethyl)ethylenediamine-N,N′,N′-triacetic acid, ethylenediamine-N,N,N′,N′-tetrakis(methylenephosponic acid), O,O′-bis(2-aminoethyl)ethyleneglycol-N,N,N′,N′-tetraacetic acid, N,N-bis(2-hydroxybenzyl)ethylenediamine-N,N-diacetic acid, 1,6-hexamethylenediamine-N,N,N′,N′-tetraacetic acid, N-(2-hydroxyethyl)iminodiacetic acid, iminodiacetic acid, 1,2-diaminopropane-N,N,N,N′-tetraacetic acid, nitrilotriacetic acid, nitrilotripropionic acid, nitrilotris(methylenephosphoric acid), 7,19,30-trioxa-1,4,10,13,16,22,27,33-octaazabicyclo[11,11,11] pentatriacontane hexahydrobromide, triethylenetetramine-N,N,N′,N″,N′″,N′″-hexaacetic acid, and combinations thereof.

### v. pH Modifying Agents

The compositions described herein can further contain sufficient amounts of at least one pH modifier to ensure that the composition has a final pH of about 3 to about 11. Suitable pH modifying agents include, but are not limited to, sodium hydroxide, citric acid, hydrochloric acid, acetic acid, phosphoric acid, succinic acid, sodium hydroxide, potassium hydroxide, ammonium hydroxide, magnesium oxide, calcium carbonate, magnesium carbonate, magnesium aluminum silicates, malic acid, potassium citrate, sodium citrate, sodium phosphate, lactic acid, gluconic acid, tartaric acid, 1,2,3,4-butane tetracarboxylic acid, fumaric acid, diethanolamine, monoethanolamine, sodium carbonate, sodium bicarbonate, triethanolamine, and combinations thereof.

### vi. Solubility Enhancers

Suitable solubility enhancing agents include solvents such as water; diols, such as propylene glycol and glycerol; mono-alcohols, such as ethanol, propanol, and higher alcohols; DMSO; dimethylformamide; N,N-dimethylacetamide; 2-pyrrolidone; N-(2-hydroxyethyl) pyrrolidone, N-methylpyrrolidone, 1-dodecylazacycloheptan-2-one and other n-substituted-alkyl-azacycloalkyl-2-ones and other n-substituted-alkyl-azacycloalkyl-2-ones (azones).

### 2. Formulations for Enteral Administration

Immunogenic ceramide compositions can be formulated for oral delivery. Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions can influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions can be prepared in liquid form, or can be in dried powder (e.g., lyophilized) form. Liposomal or proteinoid encapsulation can be used to formulate the compositions (as, for example, proteinoid microspheres reported in U.S. Pat. No. 4,925,673). Liposomal encapsulation can be used and the liposomes can be derivatized with various polymers (e.g., U.S. Pat. No. 5,013,556). See also Marshall, K. In: Modern Pharmaceutics Edited by G. S. Banker and C. T. Rhodes Chapter 10, 1979. In general, the formulation will include the peptide (or chemically modified forms thereof) and inert ingredients which protect peptide in the stomach environment, and release of the biologically active material in the intestine.

The immunogenic ceramide compositions can be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. PEGylation is a preferred chemical modification for pharmaceutical usage. Other moieties that can be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane [see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al. (1982) J. Appl. Biochem. 4:185-189].

Another embodiment provides liquid dosage forms for oral administration, including pharmaceutically acceptable emulsions, solutions, suspensions, and syrups, which can contain other components including inert diluents; wetting agents, emulsifying and suspending agents; and sweetening, flavoring, and perfuming agents.

Controlled release oral formulations may be desirable. The immunogenic ceramide compositions can be incorporated into an inert matrix which permits release by either diffusion or leaching mechanisms, e.g., gums. Slowly degenerating matrices can also be incorporated into the formulation. Another form of a controlled release is based on the Oros therapeutic system (Alza Corp.), i.e. the drug is enclosed in a semipermeable membrane which allows water to enter and push drug out through a single small opening due to osmotic effects. For oral formulations, the location of release can be the stomach, the small intestine (the duodenum, the jejunem, or the ileum), or the large intestine. Preferably, the release will avoid the deleterious effects of the stomach environment, either by protection of the peptide (or derivative) or by release of the peptide (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings can be used as mixed films.

### 3. Formulations For Topical Administration

The disclosed immunogenic ceramide compositions can be applied topically to mucosal surfaces. Topical administration can be pulmonary, or through the nasal, oral (sublingual, buccal), vaginal, or rectal mucosa.

Compositions can be delivered to the lungs while inhaling and traverse across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which can be incorporated into a tablet, gel, capsule, suspension or emulsion. Standard pharmaceutical excipients are available from any formulator. Oral formulations can be in the form of chewing gum, gel strips, tablets or lozenges.

Transdermal formulations can also be prepared. These will typically be ointments, lotions, sprays, or patches, all of which can be prepared using standard technology. Transdermal formulations will require the inclusion of penetration enhancers.

### 4. Controlled Delivery Polymeric Matrices

Immunogenic ceramide formulations disclosed herein can also be administered in controlled release formulations. Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which can be liquid or solid in nature. Unless specifically defined herein, microparticles, microspheres, and microcapsules are used interchangeably. Alternatively, the polymer can be cast as a thin slab or film, ranging from nanometers to four centimeters, a powder produced by grinding or other standard techniques, or even a gel such as a hydrogel.

Either non-biodegradable or biodegradable matrices can be used for delivery of the disclosed compositions, although biodegradable matrices are preferred. These can be natural or synthetic polymers, although synthetic polymers are preferred due to the better characterization of degradation and release profiles. The polymer is selected based on the period over which release is desired. In some cases linear release can be most useful, although in others a pulse release or “bulk release” can provide more effective results. The polymer can be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl. Polymer Sci., 35:755-774 (1988).

The devices can be formulated for local release to treat the area of implantation or injection which will typically deliver a dosage that is much less than the dosage for treatment of an entire body or systemic delivery. These can be implanted or injected subcutaneously, into the muscle, fat, or swallowed.

E. Dosage Unit Forms

The disclosed immunogenic ceramide compositions are preferably formulated in dosage unit form for ease of administration and uniformity of dosage. The expression “dosage unit form” as used herein refers to a physically discrete unit of disclosed immunogenic ceramide formulation appropriate for the subject to be treated. Animal models can be used to achieve a desirable concentration range and route of administration. Such information can then be used to determine useful doses and routes for administration in humans. Therapeutic efficacy and toxicity of the disclosed formulations can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index and it can be expressed as the ratio, LD₅₀/ED₅₀. Pharmaceutical compositions which exhibit large therapeutic indices are preferred. The data obtained from cell culture assays and animal studies can be used in formulating a range of dosages for human use.

The disclosed immunogenic ceramide compositions can be presented in unit-dose or multi-dose containers, such as sealed ampules and vials, and can be stored in a freeze-dried (lyophilized) condition requiring only the addition of the sterile liquid carrier, for example, water for injections, immediately prior to use. The immunogenic compositions can be stored at temperatures of from about 4° C. to −100° C. The immunogenic compositions can also be stored in a lyophilized state at different temperatures including room temperature. Extemporaneous injection solutions and suspensions can be prepared from sterile powders, granules and tablets commonly used by one of ordinary skill in the art. The immunogenic formulation can be sterilized through conventional means known to one of ordinary skill in the art. Such means include, but are not limited to filtration, radiation and heat. The immunogenic formulation can also be combined with bacteriostatic agents, such as thimerosal, to inhibit bacterial growth.

### III. Anti-Ceramide Antibodies

The disclosed immunogenic ceramide compositions can be used to generate antibodies that specifically bind to ceramide in vitro or in vivo. The antibodies can be monoclonal or polyclonal antibodies. Methods for producing antibodies are known in the art.

The disclosed antibodies can be xenogeneic, allogeneic, syngeneic, or modified forms thereof, such as humanized or chimeric antibodies. The term “antibody” is meant to include both intact molecules as well as fragments thereof that include the antigen-binding site and are capable of binding to a ceramide epitope. These include, Fab and F(ab′)₂ fragments which lack the Fc fragment of an intact antibody, clear more rapidly from the circulation, and may have less non-specific tissue binding than an intact antibody (Wahl et al., J. Nuc. Med. 24:316-325 (1983)). Also included are Fv fragments (Hochman, J. et al. (1973) Biochemistry 12:1130-1135; Sharon, J. et al.(1976) Biochemistry 15:1591-1594). These various fragments are produced using conventional techniques such as protease cleavage or chemical cleavage (see, e.g., Rousseaux et al., Meth. Enzymol., 121:663-69 (1986)).

Monoclonal antibodies (mAbs) and methods for their production and use are described in Kohler and Milstein, Nature 256:495-497 (1975); U.S. Pat. No. 4,376,110; Hartlow, E. et al., Antibodies: A Laboratory Manual, Cold Spring Harbor Laboratory Press, Cold Spring Harbor, N.Y., 1988); Monoclonal Antibodies and Hybridomas: A New Dimension in Biological Analyses, Plenum Press, New York, N.Y. (1980); H. Zola et al., in Monoclonal Hybridoma Antibodies: Techniques and Applications, CRC Press, 1982)).

Monoclonal antibodies can be produced using conventional hybridoma technology, such as the procedures introduced by Kohler and Milstein, Nature, 256:495-97 (1975), and modifications thereof (see above references). An animal, preferably a mouse is primed by immunization with an immunogen as above to elicit the desired antibody response in the primed animal. B lymphocytes from the lymph nodes, spleens or peripheral blood of a primed, animal are fused with myeloma cells, generally in the presence of a fusion promoting agent such as polyethylene glycol (PEG). Any of a number of murine myeloma cell lines are available for such use: the P3-NS1/1-Ag4-1, P3-x63-k0Ag8.653, Sp2/0-Ag14, or HL1-653 myeloma lines (available from the ATCC, Rockville, Md.). Subsequent steps include growth in selective medium so that unfused parental myeloma cells and donor lymphocyte cells eventually die while only the hybridoma cells survive. These are cloned and grown and their supernatants screened for the presence of antibody of the desired specificity, e.g. by immunoassay techniques using B7-DC or B7-H1 fusion proteins. Positive clones are subcloned, e.g., by limiting dilution, and the monoclonal antibodies are isolated.

Hybridomas produced according to these methods can be propagated in vitro or in vivo (in ascites fluid) using techniques known in the art (see generally Fink et al., Prog. Clin. Pathol., 9:121-33 (1984)). Generally, the individual cell line is propagated in culture and the culture medium containing high concentrations of a single monoclonal antibody can be harvested by decantation, filtration, or centrifugation.

The antibody can be produced as a single chain antibody or scFv instead of the normal multimeric structure. Single chain antibodies include the hypervariable regions from an Ig of interest and recreate the antigen binding site of the native Ig while being a fraction of the size of the intact Ig (Skerra, A. et al. Science, 240: 1038-1041 (1988); Pluckthun, A. et al. Methods Enzymol. 178: 497-515 (1989); Winter, G. et al. Nature, 349: 293-299 (1991)). In a preferred embodiment, the antibody is produced using conventional molecular biology techniques.

Polyclonal antibodies are obtained as sera from immunized animals such as rabbits, goats, rodents, etc. and can be used directly without further treatment or can be subjected to conventional enrichment or purification methods such as ammonium sulfate precipitation, ion exchange chromatography, and affinity chromatography.

### IV. Methods of Use

Extracellular ceramide can be released from cells or tissues damaged as a result of an injury or due to the existence of a disease or disorder. Extracellular ceramide can, in turn, trigger apoptotic cell death in tissue surrounding the area of acute diseased or damaged tissue, thus exacerbating the cell death and tissue damage resulting from the injury or disease. Extracellular ceramide can also be produced in response to injury or disease by the action of secretory SMAse. Methods for using the disclosed immunogenic ceramide compositions and anti-ceramide antibodies to reduce extracellular ceramide levels, to inhibit or reduce ceramide-induced cell death, and to treat or reduce the risk of developing one or more symptoms of a disease or disorder associated with ceramide-induced cell death are provided.

In one embodiment, the disclosed immunogenic ceramide compositions are administered to an individual in an effective amount to induce an immune response in a subject. The immune response induced in the subject preferably includes a humoral immune response that includes the generation of antibodies that recognize and bind to ceramide. In a preferred embodiment, the immune response induces a titer of anti-ceramide antibodies in the serum of a subject that is at least 0.1, 0.25, 0.5, 1, 2, 3, 4, 5, 10, 25, 50 or 100 μg/ml of serum.

Antibodies generated in response to administration of the disclosed immunogenic ceramide compositions are preferably effective to bind to and reduce levels of extracellular ceramide and/or to inhibit one or more biological activities of extracellular ceramide. A reduction in extracellular ceramide levels can be a systemic reduction in extracellular ceramide levels or can be a reduction in the levels of extracellular ceramide locally at the site of an injury or diseased state. A reduction in extracellular ceramide levels can result from binding by ceramide-specific antibodies and clearance of the ceramide-antibody complex through known biological mechanisms, such as binding of the complexes by Fc receptors on phagocytic or other cells. A preferred biological activity that is inhibited by ceramide-specific antibodies generated in response to the disclosed immunogenic ceramide compositions is the ability to induce apoptosis.

In another embodiment, the disclosed immunogenic ceramide compositions or anti-ceramide antibodies are administered to an individual in an effective amount to inhibit or reduce or reduce the risk of ceramide-induced cell death in a subject.

In another embodiment, the disclosed immunogenic ceramide compositions or anti-ceramide antibodies are administered to an individual in an effective amount to treat or reduce the risk of developing one or more symptoms of a disease or disorder associated with ceramide-induced cell death. The disclosed immunogenic ceramide compositions can be administered therapeutically or prophylactically. Thus, in one embodiment, treating can include directly affecting or curing, suppressing, inhibiting, preventing, reducing the risk of developing, reducing the severity of, or delaying the onset of, symptoms associated with ceramide-induced cell death, or a combination thereof.

Therapeutically effective amounts of the disclosed immunogenic ceramide formulations or anti-ceramide antibodies refers to amounts effective to delay progression, expedite remission, induce remission, augment remission, speed recovery, increase efficacy of or decrease resistance to alternative therapeutics, or a combination thereof. Therapeutically effective amounts can be effective in reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

Prophylactically effective amounts of the disclosed immunogenic ceramide compositions or anti-ceramide antibodies refers to amounts effective to delay the onset of symptoms, prevent relapse to a disease, decrease the number or frequency of relapse episodes, increasing latency between symptomatic episodes, or a combination thereof.

A. Ceramide-Related Diseases or Disorders to be Treated

Diseases and disorders associated with ceramide-induced cell death are known in the art and include diseases and disorders in which there is tissue degeneration. Degenerating tissue releases ceramide as a natural degradation product, which in turn can trigger apoptosis in tissue surrounding the area of acute diseased or damaged tissue. Exemplary diseases and disorders include, but are not limited to, pulmonary diseases and disorders, neurological diseases and disorders, cardiovascular diseases and disorders, ischemic diseases and disorders and infectious diseases.

### 1. Pulmonary Diseases and Disorders

Ceramide has been implicated in the development of several pulmonary diseases and disorders (Uhlig and Gulnins, Am. J. Resp. Crit. Care Med., 178:1100-1114 (2008)), including, but not limited to, emphysema, acute lung injury and cystic fibrosis.

### i. Emphysema

Chronic Obstructive Pulmonary Disease (“COPD”) refers to a large group of lung diseases which prevent normal respiration. Approximately 11% of the population of the United States has COPD and available data suggests that the incidence of COPD is increasing. Currently, COPD is the fourth leading cause of mortality in the United States. COPD is a disease in which the lungs are obstructed due to the presence of at least one disease selected from asthma, emphysema and chronic bronchitis. The term COPD was introduced because these conditions often co-exist and in individual cases it may be difficult to ascertain which disease is responsible for causing the lung obstruction. Clinically, COPD is diagnosed by reduced expiratory flow from the lungs that is constant over several months and in the case of chronic bronchitis persists for two or more consecutive years. The most severe manifestations of COPD typically include symptoms characteristic of emphysema.

Emphysema is a disease where the gas-exchange structures (e.g., alveoli) of the lung are destroyed, which causes inadequate oxygenation that can lead to disability and death. Anatomically, emphysema is defined by permanent airspace enlargement distal to terminal bronchioles, which is characterized by reduced lung elasticity, decreased alveolar surface area and gas exchange and alveolar destruction that results in decreased respiration. Thus, the characteristic physiological abnormalities of emphysema are reduced gas exchange and expiratory gas flow.

The major symptom of emphysema is chronic shortness of breath. Other important symptoms of emphysema include chronic cough, coloration of the skin caused by lack of oxygen, shortness of breath after minimal physical activity, and wheezing. Additional symptoms that can be associated with emphysema include vision abnormalities, dizziness, temporary cessation of respiration, anxiety, swelling, fatigue, insomnia and memory loss. Emphysema is typically diagnosed by a physical examination that shows decreased and abnormal breathing sounds, wheezing and prolonged exhalation. Pulmonary function tests, reduced oxygen levels in the blood and a chest X-ray can be used to confirm a diagnosis of emphysema.

Cigarette smoking is the most common cause of emphysema, although other environmental toxins can also contribute to alveoli destruction. The rate of lung damage can be decreased by reducing the amounts of toxins in the lung (e.g., by ceasing to smoke). However, the damaged alveolar structures are not repaired and lung function is not regained. At least four different types of emphysema have been described according to their locations in the secondary lobule: panlobar emphysema, centrilobular emphysema, distal lobular emphysema and paracicatrical emphysema.

The toxic compounds present in smoke can activate destructive processes that include the release of excessive amounts of proteases that overwhelm normal protective mechanisms, such as protease inhibitors present in the lung. The imbalance between proteases and protease inhibitors present in the lung can lead to elastin matrix destruction, elastic recoil loss, tissue damage, and continuous lung function decline.

More recent studies have now shown a direct role for ceramide in the development of emphysema. Petrache, et al. demonstrated a role for ceramide in emphysema using a rat and mouse models (Petrache, et al., Nature Medicine, 11(5):491-8 (2005). This study demonstrated elevation of lung ceramide levels in individuals with smoking-induced emphysema. Emphysema was produced in rats and mice by installation of ceramide, and ceramide-specific antibodies decreased lung ceramides and attenuated lung apoptosis in these models. This study also suggested that a feedforward mechanism mediated by activation of the secretory ASMase is involved in the development of emphysema.

### ii. Acute Lung Injury

The accumulation of experimental and clinical evidence indicates the critical role of the secretory acid SMase in the pathogenesis of acute lung injury (von Bismarck, et al., Am. J. Respir. Crit. Care Med., 177:1233-1241 (2008); von Bismarck et al., Crit. Care Med., 35:2309-2318 (2007)). For example, in an LPS model, pulmonary edema formation is attenuated by D609 (Gomel, et al., Nat. Med., 10:155-160 (2004)), pulmonary inflammation by imipramine (von Bismarck, et al., Am. J Respir. Crit. Care Med., 177:1233-1241 (2008)), and mortality by D609 (Machleidt, et al., J. Exp. Med., 184:725-733 (1996)), NB6 (Claus, et al., FASEB J., 19:1719-1721 (2005)) and in A-SMase-null mice (Haimovitz Friedman, et al., J. Exp. Med, 186:1831-1841 (1997)). In acid-induced acute lung injury, D609 treatment attenuates pulmonary edema and improves oxygenation. Finally, imipramine ameliorates edema formation and advances oxygenation in acute lung injury induced by repeated lung lavage when given together with surfactant; this beneficial effect of imipramine was remarkably long-lived and lasted for 24 hours (von Bismarck, et al., Am. J. Respir. Crit. Care Med., 177:1233-1241 (2008)).

### iii. Cystic Fibrosis

Children with cystic fibrosis (CF) very often develop infections with P. aeruginosa and once past childhood almost all patients with cystic fibrosis suffer from a chronic pneumonia with P. aeruginosa, Burkholderia cepacia, and/or S. aureus. Although the life expectancy of patients with CF has increased, these bacterial lung infections are key to the development of the disease and very often result in destruction of the lung. CF is caused by a mutation of CFTR and occurs with a frequency of 1:2,500 births, at least in Western countries. Several recent studies suggested a proinflammatory status in the lung, and possibly also other organs, of patients with CF that triggers chronic inflammation even without a bacterial or viral infection. Thus, it was shown that even noninfected Cftr-deficient mice suffer from increased IL-8 concentrations in the trachea (Weber, et al., Am. J. Physiol. Lung Cell Mol. Physiol., 281:L71-L78 (2001); Joseph, et al., Am. J. Physiol. Lung Cell Mol. Physiol., 288:L471-L479 (2005)). Further, studies on aborted embryos with CF and on BAL fluids from patients with CF as young as 4 weeks with negative cultures for CF-related bacteria, virus, and fungi, revealed a significant increase of proinflammatory mediators in the lungs (Zahm, et al., Am. J. Physiol., 272:C853-C859 (1997); Tirouvanziam, et al., Am. J. Respir. Cell Mol. Biol., 23:121-127 (2000)). These studies suggest that patients with CF suffer from an uncontrolled inflammation in the lung that might be critical for the propensity of these patients to develop infections with P. aeruginosa and other bacteria.

Additional studies imply sphingolipids (Boujaoude, et al., J. Biol. Chem., 276:35258-35264 (2001)), and in particular ceramide, as critical regulators for the development of the high sensitivity of Cftr-deficient mice to P. aeruginosa infections (Teichgraber, et al., Nat. Med, 14:382-391 (2008)). These studies demonstrated in different Cftr-deficient mouse strains that ceramide accumulation in respiratory epithelial cells and in the submucosal glands of uninfected Cftr-deficient mice is age dependent (Teichgraber, et al., Nat. Med, 14:382-391 (2008)).

### 2. Neurological Diseases and Disorders

Ceramide has also been implicated in the development of several neurological diseases and disorders (Luberto, et al., Neurochem. Res., 27:609-17 (2002)), including, but not limited to, hereditary sensory neuropathy type 1 (HSN1), stroke, Alzheimer's disease (AD), HIV-associated dementia (HAD), multiple sclerosis (MS), amyotrophic lateral sclerosis (ALS), encephalitis and Batten disease.

### i. Hereditary Sensory Neuropathy Type 1 (HSN1)

Hereditary sensory neuropathy type 1 (HSN1) is the most common hereditary disorder of peripheral sensory neurons. It is characterized by the progressive degeneration of dorsal root ganglia and motor neurons with onset during the second or third decades. Initial symptoms are sensory loss in the feet followed by distal muscle wasting and weakness. Mutations of the gene SPTLC1, encoding serine palmitoyltransferase, long chain base subunit-1 (Lcb1p subunit) have recently been identified as the cause of this disease (Bejaoui, et al., Nat. Genet., 27:261-262 (2001); Dawkins, et al., Nat. Genet., 27:309-312 (2001)), and it was postulated that these mutations augment sphingolipid-dependent apoptosis. However, a recent study demonstrated that the HSN1-like mutations in the Saccharomyces cerevisiae Lcb1p subunit were dominant inactivating mutations. These results suggest that the pathology associated with the HSNlneuropathy might result from reduced rather than increased SPT activity (Gable, et al., J. Biol. Chem., 277:10194-10200 (2002)).

### ii. Alzheimer's Disease

Alzheimer's disease (AD) is a major illness of dementia characterized histologically by the presence of amyloid plaques, neurofibrillary tangles, and extensive neuronal apoptosis. Accumulating evidence indicates elevated levels of ceramide at the very earliest clinical stage of the disease, and levels are elevated more than three-fold when compared with age-matched control (Han, et al., J. Neurochem., 82:809-818 (2002)). Subsequent studies showed that fibrillar Aβ injection in mouse increased ceramide levels in hippocampus and cortex after 7 days following injection (Alessenko, et al., Biochem. Soc. Trans., 32:144-146 (2004)) and that elevated ceramide levels increased the half-life of BACE1 and promoted Aβ biogenesis. Exogenous C6 ceramide as well as increased levels of endogenous ceramide induced by sphingomyelinase treatment promoted biogenesis of Aβ. C6 ceramide also restored Aβ generation in FB1 treated cells (Puglielli, et al., J. Biol. Chem., 278:19777-19783 (2003)).

### iii. HIV-Associated Dementia (HAD)

Human immunodeficiency virus type 1 (HIV-1) infection is known to cause CNS disorders, including HAD. Sphingolipid imbalance plays an important role in neuronal dysfunction and death in HAD. Brain tissues and CSF from patients with HAD evidenced increased oxidative stress with abnormal accumulation of sphingomyelin and ceramide (Haughey, et al., Ann. Neurol., 55:257-267 (2004)). In a separate study, it was shown that HIV-1 coat protein gp120 (glycoprotein 120) induced neuronal apoptosis in the HAD CNS through the CXCR4-NADPH oxidase-superoxide-NSMase-ceramide pathway.

### iv. Multiple Sclerosis (MS)

MS is the most common human CNS demyelinating disease, and a disorder in which oxidative stress is proposed to play an important role in oligodendroglial death though molecular mechanisms that couple oxidative stress to the oligodendrocyte losses are poorly understood. Studies have shown that the neutral sphingomyelinase-ceramide pathway is involved in mediating oxidative stress-induced apoptosis and cell death in human primary oligodendrocytes (Jana and Pahan, J. Neuroimmune Pharmacol., 2:184-193 (2007)). Lee et al. found that exogenously added bacterial sphingomyelinase exacerbated AP-induced oligodendrocyte death via an oxidative mechanism (Lee, et al., J. Cell Biol., 164:123-131 (2004)).

### v. Amyotrophic Lateral Sclerosis (ALS)

ALS is a progressive neurodegenerative disease characterized by degeneration of motor neurons in the spinal cord and producing progressive paralysis and death. Abnormal buildup of sphingomyelin, ceramide and cholesterol esters has been observed in ALS, and in the mouse model of ALS (Cu/ZnSOD mutant mice) (Cutler, et al., Ann. Neurol., 52:448-457 (2002)), and this abnormal lipid accumulations occurs in transgenic mice prior to any sign of cell death. Pharmacological blockage of sphingolipid synthesis and ceramide accumulation could suppress neuronal death via various inducers of cell death including oxidative stress (Cutler, et al., Ann. Neurol., 52:448-457 (2002)). In another study, motor neurons over-expressing the ALS-linked SOD1^{G93A} mutation showed greater susceptibility to the p75^{NTR}-activated apoptotic pathway that is associated with decreased antioxidant defenses and increased neutral sphingomyelinase activation. This apoptotic pathway is critically modulated by nuclear factor erythroid 2-related factor 2 (Nrf2) activity (Pehar, et al., J. Neurosci., 27:7777-7785 (2007)). In cerebral ischemia in vivo, increased ceramide levels have been attributed to down-regulation of glucosylceramide synthase (Yu, et al., J. Mol. Neurosa, 15:85-97 (2000); Takahashi, et al., J. Cereb. Blood Flow Metab., 24:623-627(2004); Ohtani, et al., Brain Res., 1023:31-40 (2004)).

### vi. Batten Disease

The neuronal ceroid lipofuscinoses (NCL) (Batten disease) are a group of inherited lysosomal storage diseases, and recent evidence is beginning to implicate ceramide in their pathogenesis. They are neurodegenerative diseases characterized by progressive loss of vision, seizures, cognitive decline, and early death. There is accelerated apoptosis of photoreceptors and cortical neurons. Puranam et al. have reported an increased brain ceramide level in different Batten disease types (Puranam, et al., Neuropediatrics, 28:37-41 (1997)). Eight forms of NCL have been identified and result from mutations of genes (CNL1 to CNL8) that encode for proteins involved in different aspects of lysosomal protein catabolism. Mutations in the CNL3 gene result in the juvenile NCL, and ceramide levels are increased in the brains of patients with this mutation and decreased in cells overexpressing the CLN3 protein. It has been shown that CLN3 overexpression in NT2 neuronal precursor cells protected cells from growth inhibition induced by serum starvation and protected cells from apoptosis induced by vincristine, staurosporine, and etoposide but not from death caused by exogenous ceramide (Puranam, et al., Mol. Genet. Metab., 66:294-308 (1999)). A recent study by Rylova et al. confirmed the role of CLN3 as an antiapoptotic protein as treatment with antisense to CLN3 inhibited the growth and viability of several cancer cell lines and increased ceramide levels.

### 3. Cardiovascular Diseases and Disorders

Ceramide has also been implicated in the development of several cardiovascular diseases and disorders (Pavoine and Pecker, Cardiovascular Res., 82:175-183 (2009)), including, but not limited to, atherosclerosis and heart failure.

### i. Atherosclerosis

Both proliferation and death of VSMCs contribute to the progression of the atherosclerotic lesions. Levade and colleagues were the first to reveal the possible involvement of the sphingomyelin/ceramide pathway in atherogenesis, through a mitogenic effect on VSMCs (Auge, et al., J. Biol. Chem., 271:19251-19255 (1996)). Endothelial cells, which cover the atherosclerotic lesions, secrete secretory acid SMase. Enzyme secretion is enhanced by atherogenic pro-inflammatory cytokines (Marathe, et al., J. Biol. Chem., 273:4081-4088 (1998)). Secreted acid SMase hydrolyses SM to ceramide on the surface of atherogenic lipoprotein particles, even at neutral pH (Schissel, et al. J. Biol. Chem., 273:2738-2746 (1998)). The resulting increase in lipoprotein ceramide promotes fusion and subendothelial aggregation of the lipoprotein particles, increasing their affinity for arterial wall proteoglycans and leading to foam cell formation. Studies in patients and experimental models confirm the presence of S-ASMase in atherosclerotic lesions (Marathe, et al., Arterioseler. Thromb. Vase. Biol., 19:2648-2658 (1999)), and show that the latter are significantly decreased upon pharmacological inhibition of SM synthesis.66 Also, oxidized phospholipids that are found in atherosclerotic lesions can promote VSMC death via ASMase activation (Loidl, et al., J. Biol. Chem., 278:32921-32928 (2003)). Furthermore, in a recent study using two double knockout mice models [consisting of two hyperlipidaemic models of atherosclerosis crossed onto ASMase deficient mice (producing Apoe2/2, Asm2/2 and Ldlr2/2, Asm2/2)], Tabas and colleagues showed that acid SMase deficiency reduces both lesion development and arterial trapping of atherogenic lipoproteins (Devlin, et al., Arterioscler. Thromb. Vase. Biol., 28:1723-1730 (2008)).

### ii. Heart Failure

In addition to neuro-hormonal activation, inflammation and oxidative stress are key components in chronic heart failure (HF) progression and severity. The ability of pro-inflammatory cytokines to trigger secretory acid SMase secretion from ECs (Marathe, et al., J. Biol. Chem., 273:4081-4088 (1998); Wong, et al., Proc. Natl. Acad. Sci. USA, 97:8681-8686 (2000)), combined with the stimulatory effect of reactive oxygen species (ROS) on enzyme activity, are possible mechanisms explaining the increase in plasma secretory acid SMase activity in patients with HF (DoeInter, et al., Eur. Heart J., 28:821-828 (2007)). In their study, Anker and colleagues discovered that this activity is increased by 90% in patients with HF, compared with controls, and was a significant predictor of impaired survival. Plasma secretory acid SMase activity was positively related to the disease severity (assessed by the New York Heart Association functional class and peak oxygen uptake) and main clinical markers (including creatinine, uric acid, plasma TNF-a, and sTNFR1). Impaired peripheral blood flow and vasodilator capacity are also associated with secretory acid SMase activation. This is relevant to reported increases in plasma levels of TNF-α in HF patients with impaired peripheral blood flow and the finding by Zhang, et al. (Zhang, et al., Am. J. Physiol. Heart Circ. Physiol., 283:H1785-H1794 (2002)) that desipramine neutralizes the inhibitory effect of TNF-α on endothelium-dependent vasorelaxation.

### 4. Ischemic Diseases and Disorders

Ceramide has also been implicated in the development of several ischemic diseases and disorders, including, but not limited to, ischemia/reperfusion injury, stroke and nephrotic shock.

### i. Ischemia/Reperfusion Injury

Prolonged myocardial ischaemia inevitably results in cell death, and the duration of ischaemia is a primary determinant of infarct size. Reoxygenation through reperfusion reduces ischaemic damage, but also triggers additional cell death. Preconditioning, which consists of applying transient episodes of ischaemia/reperfusion before the sustained ischaemic event, protects the heart from ischaemia/reperfusion injury by limiting apoptosis. Postconditioning has recently emerged as a more relevant clinical strategy; it consists of applying transient episodes of ischaemia/reperfusion after the sustained ischaemic event, instead of before. Pre- and postconditioning cardioprotective strategies can rely on a similar signaling pathway in the reperfused heart.

Several studies suggest a causal relationship between the increase in ceramide content and CM death in the postischaemic reperfused rat heart (Bielawska, et al., Am. J. Pathol., 151:1257-1263 (1997); Cordis, et al., J. Pharm. Biomed. Anal., 16:1189-1193 (1998); Beresewicz, et al., J. Physiol. Pharmacol., 53:371-382 (2002)). Argaud, et al. have shown that benefits of preconditioning are related to reduced-cardiac ceramide content (Argaud, et al., Am. J. Physiol. Heart Circ. Physiol., 286:H246-H251 (2004)). The acid SMase inhibitor, tricyclodecan-9-yl-xanthate (D609), administered before the ischaemic period, reproduces preconditioning protection, proving the contribution of ASMase activity in the ischaemia-induced cell death. However, Lecour et al. report that preconditioning with TNF-α, that is likely to activate acid SMase and/or neutral SMase, also exerts an ischaemic preconditioning-like protection (Lecour, et al., J. Mol. Cell. Cardiol., 34:509-518 (2002)). TNF-a protection is reproduced by the cell-permeable C2-ceramide. The discrepancy between these two reports probably illustrates the multiple responses that ceramide can mediate depending on its subcellular location, which determines its proximal targets and downstream metabolism. It may be that acid SMase activation triggered by the ischaemic preconditioning provides ceramide integral to a cell death pathway, whereas TNF-a and cell permeable C2-ceramide release ceramide for the ceramidase/sphingosine kinase metabolism cascade. In fact, the ceramidase inhibitor N-oleoylethanolamine hinders the preconditioning-like protection provided by TNF-a or C2-ceramide, but does not hinder the protection induced by ischaemic preconditioning.

Using the tricyclic antidepressant inhibitor desipramine (a potent ASMase inhibitor), Das and co-workers document the two-edged role of ceramide, mediating protection in ischaemic preconditioning but promoting apoptosis after the ischaemia/reperfusion event (Cui, et al., J. Am. Coll. Surg., 198:770-777 (2004); Der, et al., J. Mol. Cell. Cardiol., 40:313-320 (2006)). Thus, ASMase-mediated accumulation of ceramide in the ischaemic heart is causally related with apoptosis and cardiac dysfunction.

### ii. Stroke

Ischemic stroke is a major cause of disability. Ceramide levels are known to increase in ischemic injury (Toman, et al., J. Neurotrauma, 17:891-898 (2000)), and it was recently reported that levels of acid SMase were highly increased in mice subjected to transient focal cerebral ischemia. This resulted in the generation of ceramide and the production of inflammatory cytokines. The extent of brain tissue damage was decreased and behavioral outcome improved in mice lacking acid SMase and in wild-type mice treated with an inhibitor of acid SMase (Yu, et al., J. Mol. Neurosci., 15:85-97 (2001)). The immunosuppressant FK506 has also been shown to inhibit ceramide generation and apoptosis in rats with ischemic stroke (Herr, et al., Brain Res., 826:210-219 (1999)).

### 5. Infectious Diseases

Ceramide has also been implicated in deleterious effects of infectious diseases, including, but not limited to, sepsis.

### i. Sepsis

One of the early documented effects of acid SMase was in LPS-induced apoptosis, when it was shown that wild-type mice injected with LPS had serum ASM activity that was increased 2- to 2.5-fold (Wong, et al., Proc. Natl. Acad. Sci. U.S.A., 97:8681-8686 (2000)). This finding suggested that acid SMase can play a role in sepsis and that inhibition of serum acid SMAse should be considered as a therapeutic approach for certain infections. Recently, the specific role of acid SMAse in LPS signaling has been further elucidated. The LPS response by macrophages requires activation of the Toll-like receptor 4 (TLR4) complex, which itself requires ceramide-rich lipid microdomains to assemble. Notably, pharmacologic inhibition of acid SMAse prevented TLR4 complex formation after LPS administration, and exogenous ceramide rescued this inhibition (Cuschieri, et al., Surg. Infect. (Larchmt.), 8:91-106 (2007)). These observations, in addition to others, suggest a role for acid SMAse in sepsis.

B. Methods of Administration

The disclosed immunogenic ceramide composiotions or anti-ceramide antibodies can be administered before, during or after the onset of symptoms associated with a disease or disorder associated with ceramide-induced cell death. Any acceptable method known to one of ordinary skill in the art can be used to administer the disclosed immunogenic ceramide compositions or anti-ceramide antibodies to a subject. In general, methods of administering immunogenic compositions and antibodies are well known in the art.

The administration can be localized (i.e., to a particular region, physiological system, tissue, organ, or cell type) or systemic. Immunogenic ceramide compositions can be administered by different routes, such as oral, including buccal and sublingual, rectal, parenteral, aerosol, nasal, intramuscular, subcutaneous, intradermal, intravenous, intraperitoneal, and topical. The immunogenic composition can also be administered in the vicinity of lymphatic tissue, for example through administration to the lymph nodes such as axillary, inguinal or cervical lymph nodes, or to the spleen or mucosal-associated lymphoid tissue. In some embodiments, the immunogenic composition can be injected at multiple locations. The particular route of administration selected will depend upon factors such as the particular formulation, the severity of the state of the subject being treated, and the dosage required to induce an effective immune response.

The disclosed immunogenic ceramide formulations can be administered in different forms, including but not limited to solutions, emulsions and suspensions, microspheres, particles, microparticles, nanoparticles, and liposomes.

### 1. Effective Dosages of Immunogenic Ceramide Compositions

The actual effective amounts of immunogenic ceramide compositions can vary according to factors including the specific immunogenic ceramides or combinations thereof being utilized, the concentration and/or nature of associated carrier molecules and additional adjuvants, the mode of administration, and the age, weight, condition of the subject being treated, as well as the route of administration and the disease or disorder.

An effective amount of the immunogenic composition can be ideally obtained after one single administration. In certain circumstances, especially for the elderly population, or in the case of young children (below 9 years of age) who are vaccinated for the first time against a particular antigen, it can be beneficial to administer two doses of the same composition. The second dose of the same composition (still considered as composition for first vaccination) can be administered during the on-going primary immune response and is adequately spaced in time from the first dose. Typically the second dose of the composition is given a few weeks, or about one month, e.g. 2 weeks, 3 weeks, 4 weeks, 5 weeks, or 6 weeks after the first dose, to help prime the immune system in unresponsive or poorly responsive individuals.

In a specific embodiment, the administration of the immunogenic ceramide composition alternatively or additionally induces a B-memory cell response in subjects that results in an increased frequency of peripheral blood B lymphocytes capable of differentiation into antibody-secreting plasma cells upon antigen encounter as measured by stimulation of in vitro differentiation. The administration of a single dose of the immunogenic composition for first vaccination provides better sero-protection and induces an improved CD4 T-cell, or CD8 T-cell immune response against a specific antigen compared to that obtained with the un-adjuvanted formulation. This improved response can be especially beneficial in an immuno-compromised human population such as the elderly population (65 years of age and above) and in particular the high risk elderly population. This can result in reducing the overall morbidity and mortality rate and preventing emergency admissions to hospital for pneumonia and other influenza-like illness. This can also be of benefit to the infant population (below 5 years, preferably below 2 years of age). Furthermore it allows inducing a CD4 T cell response which is more persistent in time, e.g. still present one year after the first vaccination, compared to the response induced with the un-adjuvanted formulation.

Preferably the CD4 T-cell immune response, such as the improved CD4 T-cell immune response obtained in an unprimed subject, involves the induction of a cross-reactive CD4 T helper response. In particular, the amount of cross-reactive CD4 T cells is increased. The term “cross-reactive” CD4 response refers to CD4 T-cell targeting shared epitopes for example between influenza strains.

The dose of immunogenic ceramide or ceramide analog is suitably able to induce an immune response to ceramide in a human. Usually an immunogenic composition dose will range from about 0.5 ml to about 1 ml. Typical vaccine doses are 0.5 ml, 0.6 ml, 0.7 ml, 0.8 ml, 0.9 ml or 1 ml. In a preferred embodiment, a final concentration of 50 μg of ceramide or ceramide analog, is contained per ml of vaccine composition, or 25 μg per 0.5 ml vaccine dose. In other preferred embodiments, final concentrations of 35.7 μg or 71.4 μg of ceramide or ceramide analog is contained per ml of vaccine composition. Specifically, a 0.5 ml vaccine dose volume contains 25 μg or 50 μg of ceramide or ceramide analog per dose. In still another embodiment, the dose is 100 μg or more.

### 2. Revaccination (Boosting Formulation)

Subjects can be revaccinated with the immunogenic ceramide formulations. Typically revaccination is made at least 6 months after the first vaccination(s), preferably S to 14 months after, more preferably at around 10 to 12 months after.

Preferably revaccination induces any, preferably two or all, of the following: (i) an improved effector cell response against the antigenic preparation, or (ii) an improved B cell memory response or (iii) an improved humoral response, compared to the equivalent response induced after a first vaccination with the formulation.

### 3. Vaccination Devices

Any suitable device can be used for intradermal delivery, for example short needle devices. Intradermal vaccines can also be administered by devices which limit the effective penetration length of a needle into the skin. Jet injection devices which deliver liquid vaccines to the dermis via a liquid jet injector or via a needle which pierces the stratum corneum and produces a jet which reaches the deunis can also be used. Jet injection devices are known in the art. Ballistic powder/particle delivery devices which use compressed gas to accelerate vaccine in powder form through the outer layers of the skin to the dermis can also be used. Additionally, conventional syringes can be used in the classical Mantoux method of intradermal administration.

Another suitable administration route is the subcutaneous route. Any suitable device can be used for subcutaneous delivery, for example classical needle. Preferably, a needle-free jet injector service is used. Needle-free injectors are known in the art. More preferably the device is pre-filled with the liquid vaccine formulation.

Alternatively the vaccine is administered intranasally. Typically, the vaccine is administered locally to the nasopharyngeal area, preferably without being inhaled into the lungs. It is desirable to use an intranasal delivery device which delivers the vaccine formulation to the nasopharyngeal area, without or substantially without it entering the lungs. Preferred devices for intranasal administration of vaccines are spray devices. Nasal spray devices are commercially available. Nebulizers produce a very fine spray which can be easily inhaled into the lungs and therefore does not efficiently reach the nasal mucosa. Nebulizers are therefore not preferred. Preferred spray devices for intranasal use are devices for which the performance of the device is not dependent upon the pressure applied by the user. These devices are known as pressure threshold devices. Liquid is released from the nozzle only when a threshold pressure is applied. These devices make it easier to achieve a spray with a regular droplet size. Pressure threshold devices suitable for use with the present invention are known in the art and are commercially available.

Preferred intranasal devices produce droplets (measured using water as the liquid) in the range 1 to 200 μm, preferably 10 to 120 μm. Below 10 inn there is a risk of inhalation, therefore it is desirable to have no more than about 5% of droplets below 10 μm. Droplets above 120 μm do not spread as well as smaller droplets, so it is desirable to have no more than about 5% of droplets exceeding 120 μm.

Bi-dose delivery is another feature of an intranasal delivery system for use with the vaccines. Bi-dose devices contain two sub-doses of a single vaccine dose, one sub-dose for administration to each nostril. Generally, the two sub-doses are present in a single chamber and the construction of the device allows the efficient delivery of a single sub-dose at a time. Alternatively, a monodose device can be used for administering the vaccines.

Alternatively, the epidermal or transdermal vaccination route is also contemplated.

In a specific aspect, the immunogenic formulation for the first administration can be given intramuscularly, and the boosting composition, can be administered through a different route, for example intradermal, subcutaneous or intranasal.

### 4. Exemplary Vaccination Regimes and Dosing

In one embodiment, the immunogenic formulations can be a standard 0.5 ml injectable dose in most cases, containing 15 μg of immunogenic ceramide or ceramide analog. The vaccine dose volume can be between 0.5 ml and 1 ml, in particular a standard 0.5 ml, or 0.7 ml vaccine dose volume. Slight adaptation of the dose volume will be made as needed.

A lower dose vaccine can be provided in a smaller volume than the conventional injected vaccines, which are generally around 0.5, 0.7 or 1 ml per dose. The low volume doses according to the invention are preferably below 500 μl, more preferably below 300 μl and most preferably not more than about 200 μl or less per dose. Thus, a preferred low volume vaccine dose is a dose with a low antigen dose in a low volume, e.g. about 15 μg or about 7.5 μg antigen or about 3.0 μg antigen in a volume of about 200 μl.

D. Combination Therapy

The disclosed immunogenic ceramide compositions or anti-ceramide antibodies can be administered alone or in combination with one or more additional therapeutic or prophylactic agents, or can be coupled with surgical, radiologic, or other approaches in order to affect treatment. For example, the disclosed immunogenic compositions can be administered in combination with one or more anti-inflammatory or anti-apoptotic agents.

### 1. Anti-Inflammatory Agents

Anti-inflammatory agents can be non-steroidal, steroidal, or a combination thereof. Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents can also be employed.

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

### 2. Anti-Apoptotic Agents

Representative examples of anti-apoptotic agents include, without limitation, cyclocreatine, cyclocreatine phosphate, acetyl L-carnitine, coenzyme Q10, glutathione, or a-lipoic acid, caspase inhibitors (e.g., fluoromethylketone peptide derivatives), calpain inhibitors, cathepsin inhibitors, nitric oxide synthase inhibitors, flavonoids, vitamin A, vitamin C, vitamin E, vitamin D, pycnogenol, super oxidedismutase, N-acetyl cysteine, selenium, catechins, alpha lipoic acid, melatonin, glutathione, zinc chelators, calcium chelators, and L-arginine. Additional anti-apoptotic agents include mitochondrial proteins or peptides thereof that inhibit apoptosis such as Bcl-2, Bcl-XL and Bcl-XW.

### EXAMPLES

### Example 1

### Generation of a Ceramide-Specific Rabbit IgG Antibody

Materials and Methods:

Materials

New Zealand White female rabbits were purchased from Myrtle's Rabbitry (Thompson Station, Tenn.). D-erythro-C18 ceramide was from Matreya (Pleasant Gap, Pa.). Hoechst 33258, myriocin, HRP-conjugated anti-rabbit IgG, keyhole limpet hemocyanin (KLH), and SIGMA FASTio-phenylenediamine dihydroehloride peroxidase substrate tablets were from Sigma-Aldrich (St. Louis, Mo.). Immulon 1B flat-bottomed 96-well microtiter plates were from Thermo Electron Corp. (Milford, Mass.). SphingoStripsi, Alexa Fluor: 647-conjugated phalloidin, and Alexa Fluor: 594-conjugated wheat germ agglutinin were obtained from Invitrogen (Carlsbad, Calif.). Cy3-conjugated donkey anti-rabbit IgG, Cy2-conjugated donkey anti-rabbit IgG, Cy2-conjugated donkey antimouseIgM, m-chain-specific and normal donkey serum were purchased from Jackson ImmunoResearch (West Grove, Pa.). The polyclonal mouse anti-ceramide IgM MAS0020 was from Glycobiotech (Kukels, Germany). Bacterial sphingomyelinase was from Calbiochem (San Diego, Calif.). Blocking-grade dry milk and nitrocellulose membranes were from Bio-Rad (Hercules, Calif.).

Immunization of Rabbits

C18 ceramide (2 mg) was dissolved in chloroform-methanol (2:1, v/v) and dried under a steady stream of nitrogen. One milliliter of KLH (2 mg/ml) in PBS was added to the dried residue. This was mixed with an equal volume of

Freund's Complete Adjuvant to form an emulsion. One milliliter of the emulsion (about 1 mg of ceramide) was injected subcutaneously into the flanks of one rabbit. Booster doses were injected at 2, 4, 8, and 10 weeks after the initial injection using Freund's Incomplete Adjuvant. A small volume of blood (2-5 ml) was collected by ear-vein puncture of the rabbit at a definite time interval to determine the titer of the antiserum. When the desired titer was obtained, the rabbit was anesthetized and bled through cardiac puncture and the serum was collected from clotted blood. All procedures involving animals were conducted in conformity with the Public Health Service Policy on Humane Care and Use of Laboratory Animals, incorporated in the Institute for Laboratory Animal Research Guide for Care and Use of Laboratory Animals. Furthermore, these procedures were performed in compliance with the guidelines issued by the Committee on Animal Use for Research and Education at the Medical College of Georgia.

Purification of Anti-Ceramide Antibody

ELISA was used to determine the titer of the antiserum. Briefly, Immulon 1B flatbottomed 96-well microtiter plates were coated with 500 ng of C18 ceramide in ethanol. After washing off unbound ceramide, the reaction was blocked with 1% BSA in PBS. The wells were then incubated with various dilutions (1:50 to 1:400) of immunized and preimmune serum in 1% BSA/PBS at 37° C. for 1 hour. The unbound antibody was removed by repeated washing with PBS. HRP-conjugated anti-rabbit IgG secondary antibody (1:2,000) was added to the plate and incubated for 37° C. for 1 h. After thorough washing, the wells were incubated with o-phenylenediamine dihydrochloride peroxidase substrate for 5-15 min and the reaction was terminated using 3 N H₂SO₄. The absorbance was measured at 492 nm. To purify the IgG fraction, the total immunoglobulin from serum was precipitated using 45% ammonium sulfate and stirred overnight at 4° C. The pellet was collected by centrifugation, dissolved in a defined volume of PBS, and dialyzed against PBS to remove the ammonium sulfate with several changes of PBS. The solution after dialysis was further clarified by centrifugation, mixed with 0.02% azide, and preserved at −20° C. for further purification. To purify the IgG fraction, a portion of the immunoglobulin solution was passed through a protein A column and the eluate (citrate buffer elution) was concentrated with polyethylene glycol. Any potential anti-KLH antibody was removed by passing the IgG fraction through a gel column containing KLH immobilized on agarose. For long-term storage, the IgG fraction was mixed with glycerol (1:1), divided into aliquots, and stored at −20° C.

Lipid Overlay Assay

Lipid overlay assays were performed using either Sphingo-Stripsi or by spotting lipids on a nitrocellulose membrane. Briefly, lipids were dissolved in chloroform-methanol (2:1, v/v), spotted on the nitrocellulose membrane, and allowed to dry at room temperature for 30 minutes. The membrane was blocked with 10% dry milk in PBS. Membranes were incubated with the anti-ceramide antibodies diluted in the blocking buffer at 4° C. overnight with gentle shaking. Membranes were washed five times with PBS with vigorous shaking at room temperature and then incubated with HRP-conjugated secondary antibodies for 2-3 h at room temperature. The membranes were washed five times with PBS 1 0.2% Tween-20 with vigorous shaking at room temperature. Antibody binding was detected using a chemiluminescence system and exposure to X-ray film.

Results:

The antiserum and purified rabbit IgG fraction was tested in lipid ELISA and overlay assays using lipid-coated Immulon 1B 96-well plates, nitrocellulose membranes, and SphingoStrips. The 96-well plates were coated with 500 ng of ceramide or other lipid species per well. The staining reaction of the preimmune serum was equivalent to that of the negative control without serum and was less than 15% of the positive reaction. A positive reaction was found for C18 ceramide, the ceramide species used for immunization of the rabbits

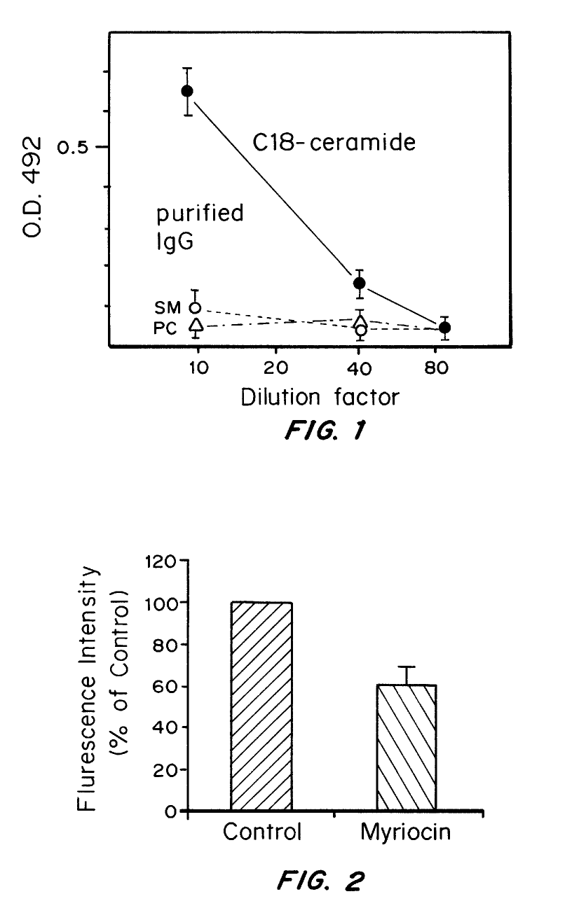

(FIG. 1). The anti-ceramide IgG fraction, however, did not react significantly with sphingomyelin or phosphatidylcholine. To use an assay method with higher sensitivity, 1.0 or 10 nmol of different ceramide species was spotted on nitrocellulose followed by lipid overlay immunostaining. The rabbit IgG recognized C16, C18, C20, and C24 ceramide in a dose dependent manner. Consistent with the results from the lipid ELISA, the strongest reaction was seen with C18 ceramide. The antibody also recognized C2 ceramide, but only at higher concentrations.

To confirm the specificity of the anti-ceramide antibody, the lipid overlay assay was performed with Sphingo-Strips, commercially available membranes spotted with 100 pmol of various sphingolipids and related lipids. The rabbit IgG fraction reacted strongly and specifically with ceramide. No reactivity was detected with phosphatidylcholine or sphingomyelin. This specificity was consistent with that of the polyclonal IgM anti-ceramide antibody that has been shown to recognize ceramide in a similar assay (Cowart, et al., J. Lipid Res., 43: 2042-2048 (2002)). As with the anti-ceramide IgM, the rabbit anti-ceramide IgG detected dihydroceramide of appropriate fatty acid chain length. Anti-ceramide rabbit IgG also reacted with phytoceramide, although significantly less intensely than with ceramide. Together, these results indicate that the rabbit IgG fraction specifically recognizes ceramide in lipid overlay assays, similar to the polyclonal mouse IgM antibody, and is capable of detecting ceramide species with different fatty acid chain lengths.

### Example 2

### Immunocytochemistry Using the Ceramide-Specific Rabbit IgG Antibody

Materials and Methods:

Immunocytochemistry and Flow Cytometry

For immunocytochemistry, F11 cells were grown on coverslips. Cells were incubated with myriocin for 3 days to inhibit de novo ceramide biosynthesis and reduce ceramide levels. To determine whether the antibody would cross-react with glycosphingolipids in the membrane, cells were incubated for 4 days with 250 mM N-butyl-deoxynojirimycin (NB-dNJ), a glucosylceramide (GlcCer) synthase inhibitor known to inhibit glycosphingolipid biosynthesis. Control and myriocin- or NB-dNJ-treated cells were fixed with 4% p-formaldehyde in PBS for 20 min at room temperature. To release endogenous ceramide from sphingomyelin, fixed cells were incubated for 1 hour at 37° C. with 0.3 units (1 U/ml) of Staphylococcus aureus sphingomyelinase. Immunocytochemistry for ceramide was performed without the use of detergent for permeabilization. The immunostaining of fixed cells or embryonic brain sections followed procedures described previously (Wang, et al., J. Biol. Chem., 280:26415-26424 (2005)), using a blocking solution of 3% ovalbumin and 2% donkey serum in PBS and primary and secondary antibodies diluted in 0.1% ovalbumin in PBS. Epifluorescence microscopy was performed with an Axiophot microscope (Carl Zeiss Microlmaging, Inc.) equipped with a Spot II charge-coupled device camera. Confocal fluorescence microscopy was performed using a Zeiss LSM510 confocal laser scanning microscope equipped with a two photon argon laser at 488 nm (Cy2), 543 inn (Cy3), or 633 nm (Alexa Fluor: 647). For flow cytometry analysis, control and myriocin-incubated F11 cells were trypsinized and passed through a 40 mm mesh. The cells were resuspended in 100 ml of blocking buffer (3% ovalbumin in PBS) and incubated at room temperature for 15 minutes. The cells were then stained with the anti-ceramide rabbit IgG antibody diluted in 0.5% ovalbumin in PBS at 4° C. for 1 hour. After washing with PBS three times, cells were stained with Cy2-conjugated anti-rabbit IgG secondary antibody at 4° C. for 1 hour. Cells were washed with PBS, and stained cells were analyzed by flow cytometry, measuring the fluorescence emission at 530 nm. The results from three independent labeling experiments were normalized against the control and represented as bar graphs.

Results:

To determine whether the rabbit IgG fraction could be used for immunocytochemistry, F11 neuroblastoma cells were stained using both the rabbit IgG fraction and the polyclonal IgM antibody for ceramide. The cells were fixed but not permeabilized before staining, because permeabilization could affect the distribution of lipids in the membrane. At lower dilution of the primary antibody (1:50), both antibodies bound to the same regions on the plasma membrane, indicated by the appearance of yellow pseudocolor in the overlay. At higher dilutions of the primary antibody (1:200), the staining with rabbit IgG was still visible, whereas the signal from the polyclonal IgM was greatly diminished, indicating that the rabbit IgG antibody was more sensitive or present at a higher concentration. When cells were incubated with myriocin (an inhibitor of de novo ceramide biosynthesis) for 3 days, the intensity of the signal was reduced with both antibodies. Incubation with myriocin is known to reduce ceramide levels in cells, consistent with diminished staining using the two antibodies. To quantify this effect, control and myriocin-incubated F11 cells were stained with the rabbit IgG fraction and analyzed by flow cytometry. FIG. 2 shows that the fluorescence intensity of the myriocin-incubated cells was reduced by about 40% compared with that of control cells. This was consistent with the observation that myriocin incubation of F11 cells for 3 days reduced ceramide levels by about 50%, as determined by high-performance thin-layer chromatography.

On the other hand, when cells were incubated with NB-dNJ (an inhibitor of GlcCer synthase), the intensity of the signal was not changed significantly. GlcCer synthase is the first enzyme in glycosphingolipid biosynthesis and uses ceramide as a substrate. Inhibiting this enzyme has been shown to reduce glycosphingolipid levels without altering ceramide levels. If the antibody were to cross-react with glycosphingolipids, a reduction in the staining intensity would be expected. The absence of such a reduction indicated that this antibody was specific for ceramide. This specificity was also confirmed by the absence of a fluorescence signal when using the IgG fraction preadsorbed to ceramide. To further test the specificity of the rabbit IgG, F11 cells were incubated with bacterial sphingomyelinase before performing immunocytochemistry. Sphingomyelinase is a phospholipase C-like enzyme that hydrolyzes sphingomyelin to release phosphorylcholine and ceramide. Incubation with bacterial sphingomyelinase has been shown to increase ceramide levels at the plasma membrane. Sphingomyelinase-incubated cells contained increased ceramide levels, as detected by the rabbit IgG fraction compared with control cells.

The distribution of ceramide on the plasma membrane and within cells was tested in a series of highresolution fluorescence analyses using F11 cells and the anti-ceramide rabbit IgG fraction. Ceramide was enriched in protrusions of the cell membrane and in a perinuclear region that was identified as the Golgi apparatus using costaining with fluorescence labeled wheat germ agglutinin. Remarkably, the anti-ceramide rabbit IgG was also able to detect ceramide in the plasma membrane of neurons of the developing cortical plate and intermediate zone. The mouse IgM stained ceramide in a similar tissue complex, although mainly in the nuclear region of cortical plate cells.

Although the presence of ceramide in neuronal nuclei is not a priori excluded, it appears to be more likely that the rabbit IgG detected the main cellular distribution site of ceramide (plasma membrane). Together, these results indicate that the rabbit IgG fraction specifically recognizes ceramide in fixed cells and tissues and therefore is suitable for the immunocytochemical detection of ceramide.

1. A method for reducing levels of extracellular ceramide or inhibiting one or more biological functions of extracellular ceramide in a subject comprising administering to a subject in need thereof an immunogenic composition comprising immunogenic ceramide and, optionally, a pharmaceutically acceptable excipient in an effective amount to induce the production of antibodies in the subject to bind to and reduce levels of extracellular ceramide or inhibit one or more biological functions of extracellular ceramide.

2. A method for reducing ceramide-induced cell death in a subject comprising administering to a subject in need thereof an immunogenic composition comprising immunogenic ceramide and, optionally, a pharmaceutically acceptable excipient in an effective amount to reduce or inhibit ceramide-induced cell death in the subject.

3. A method of treating or preventing one or more symptoms of a disease or disorder associated with ceramide-induced cell death comprising administering to a subject in need thereof an immunogenic composition comprising immunogenic ceramide, and, optionally, a pharmaceutically acceptable excipient in an effective amount to induce an immune response in the subject resulting in the treatment or reduction of one or more symptoms of a disease or disorder associated with ceramide-induced cell death.

4. The method of claim 3, wherein the disease or disorder associated with ceramide-induced cell death is selected from the group consisting of pulmonary diseases and disorders, neurological diseases and disorders, cardiovascular diseases and disorders, ischemic diseases and disorders and infectious diseases.

5. The method of claim 3, wherein the immunogenic ceramide comprises a naturally occurring ceramide, a ceramide analog, or a combination thereof, wherein the naturally occurring ceramide, ceramide analog, or combination thereof are modified either chemically or by association with an immunogenic carrier molecule to have increased immunogeneicity as compared to the ceramide in the absence of the modification.

6. The method of claim 3, wherein the immunogenic ceramide comprises naturally occurring ceramide, a ceramide analog, or a combination thereof, attached to an immunogenic carrier molecule selected from the group consisting of keyhole limpet hemocyanin (KLH), serum albumin, ovalbumin, thyroglobulin, toxoids derived from diphtheria and tetanus, bacteria outer membrane proteins, crystalline bacterial cell surface layers, gamma globulin, exotoxin A, LT toxin, Cholera B toxin, Klebsiella pneumoniae OmpA, Bacterial flagella, Clostridium difficile recombinant toxin A, peptide dendrimers (multiple antigenic peptides), and pan DR epitope (PADRE).

7. The method of claim 3, wherein the composition further comprises an adjuvant selected from the group consisting of oil emulsions; saponin formulations; virosomes and viral-like particles; bacterial and microbial derivatives; immunostimulatory oligonucleotides; ADP-ribosylating toxins and detoxified derivatives; alum; bacille Calmette-Guerin (BCG); mineral-containing compositions; bioadhesives and/or mucoadhesives; microparticles; liposomes; polyoxyethylene ether and polyoxyethylene ester formulations; polyphosphazene; muramyl peptides; imidazoquinolone compounds; surface active substances, cytokines, interleukins, interferons, macrophage colony stimulating factor, tumor necrosis factor and polypeptides of the B7 family of costimulatory molecules.

8. The method of claim 3, wherein the immunogenic composition is administered parenterally.

9. The method of claim 8, wherein the immunogenic formulation is administered subcutaneously.

10. The method of claim 3, wherein the immune response induced in the subject comprises a humoral immune response resulting in the generation of anti-ceramide antibodies.

11. The method of claim 10, wherein the anti-ceramide antibodies are produced in a titer of at least 0.1, 0.25, 0.5, 1, 2, 3, 4, 5, 10, 25, 50 or 100 μg/ml of serum.

12. The method of claim 3, wherein the immunogenic composition is administered in combination with one or more anti-inflammatory or anti-apoptotic agents.

13. An immunogenic ceramide composition for reducing levels of extracellular ceramide or inhibiting one or more biological functions of extracellular ceramide in a subject comprising an effective amount of immunogenic ceramide to induce the production of antibodies in the subject effective to bind to and reduce levels of extracellular ceramide or inhibit one or more biological functions of extracellular ceramide, and, optionally, a pharmaceutically acceptable excipient.

14. An immunogenic ceramide composition for reducing ceramide-induced cell death in a subject comprising an effective amount of immunogenic ceramide to reduce or inhibit ceramide-induced cell death in the subject, and, optionally, a pharmaceutically acceptable excipient.

15. An immunogenic ceramide composition for treating or preventing one or more symptoms of a disease or disorder associated with associated with ceramide-induced cell death comprising an effective amount of immunogenic ceramide to induce an immune response in the subject resulting in the treatment or prophylaxis of one or more symptoms of a disease or disorder associated with ceramide-induced cell death and, optionally, pharmaceutically acceptable excipients.

16. The immunogenic composition of claim 15, wherein the disease or disorder associated with ceramide-induced cell death is selected from the group consisting of pulmonary diseases and disorders, neurological diseases and disorders, cardiovascular diseases and disorders, ischemic diseases and disorders and infectious diseases.

17. The immunogenic composition of claim 13, wherein the immunogenic ceramide is ceramide or a ceramide analog attached to a carrier molecule selected from the group consisting of keyhole limpet hemocyanin (KLH), serum albumin, ovalbumin, thyroglobulin, toxoids derived from diphtheria and tetanus, bacteria outer membrane proteins, crystalline bacterial cell surface layers, gamma globulin, exotoxin A, LT toxin, Cholera B toxin, Klebsiella pneumoniae OmpA, Bacterial flagella, Clostridium difficile recombinant toxin A, peptide dendrimers (multiple antigenic peptides), and pan DR epitope (PADRE).

18. The immunogenic composition of claim 17, wherein the carrier molecule is keyhole limpet hemocyanin (KLH).

19. The immunogenic composition of claim 13 further comprising an adjuvant selected from the group consisting of oil emulsions; saponin formulations; virosomes and viral-like particles; bacterial and microbial derivatives; immunostimulatory oligonucleotides; ADP-ribosylating toxins and detoxified derivatives; alum; bacille Calmette-Guerin (BCG); mineral-containing compositions; bioadhesives and/or mucoadhesives; microparticles; liposomes; polyoxyethylene ether and polyoxyethylene ester formulations; polyphosphazene; muramyl peptides; imidazoquinolone compounds; surface active substances, cytokines, interleukins, interferons, macrophage colony stimulating factor, tumor necrosis factor and polypeptides of the B7 family of costimulatory molecules.

20. Use of the immunogenic ceramide composition of claim 13 for use in the treatment of a disease or disorder associated with ceramide-induced cell death.
